(19) United States
(12) Patent Application Publication
(10) Pub. No.: US 2018/0112316 A1
(43) Pub. Date: Apr. 26, 2018

(54) METHOD FOR THE SURFACE TREATMENT OF A BIOCORRODABLE IMPLANT

(71) Applicants: Volkmar Neubert, Clausthal-Zellerfeld (DE); Volkmar-Dirk Neubert, Clausthal-Zellerfeld (DE)

(72) Inventors: Volkmar Neubert, Clausthal-Zellerfeld (DE); Volkmar-Dirk Neubert, Clausthal-Zellerfeld (DE)

(73) Assignee: Syntellix AG, Hannover (DE)

(21) Appl. No.: 15/332,817

(22) Filed: Oct. 24, 2016

(51) Int. Cl. C23F 13/04; C23F 13/00; C22C 23/06; A61L 27/58; A61L 27/04; A61L 27/30

(52) U.S. Cl. CPC C23F 13/04; C23F 13/005; C22C 23/06; A61L 2420/02; A61L 27/047; A61L 27/306; A61L 27/58

(57) ABSTRACT

The present invention relates to a method for the surface treatment of a biocorrodable implant by means of alternating cathodic and anodic polarization, and also to a corresponding implant.

The present invention relates to a method for the surface treatment of a biocorrodable implant by means of alternating cathodic and anodic polarization, and also to a corresponding implant.

The purpose of implants is to support or to replace body functions and the most varied embodiments of implants have been used in medical technology. In addition to implants for securing tissues, endovascular implants, dental prosthesis implants, joint replacement implants, implants are also used for the treatment of bone damage, such as screws, nails, plates, or as bone replacements.

Nowadays implants which are applied to the bone are generally manufactured from titanium. In spite of the relatively good biocompatibility of titanium implants by comparison with other permanent implants, efforts have been made to further improve these titanium implants. Coatings are often provided on the surface of the implant in order to improve the biocompatibility.

One disadvantage of the application of layers to the surface of the implant is a change to the geometry of the implant, even in the event of small layer thicknesses. Moreover, the adhesion of the applied layers is generally not optimal.

DE 195 04 386 C2 discloses a method for producing a graduated coating of calcium phosphate phases and metal oxide phases on metal implants, preferably made of titanium. In the electrochemical method using a substrate electrode formed by the metal implant and a counterelectrode, an aqueous solution with calcium and phosphate ions in the weakly acidic to neutral range is used as electrolyte and the substrate electrode formed by the implant is alternately polarized anodically and cathodically. Good growth of the bone onto the implant is achieved by the firm incorporation of calcium phosphate phases into the implant surface.

DE 100 29 520 A1 describes a coating for a metal implant surface for improvement of the osteointegration. In an electrolysis cell the implant is cathodically polarized in an electrolyte containing calcium, phosphate and collagen. A mineralized collagen layer is formed on the surface of the implant by the method.

It is often only necessary for the implant to remain temporarily in the body, in particular in the case of cardiovascular and orthopedic implants. Implants made of a permanent material must then be removed by a further operation. For this reason, biocorrodable materials are used for implants. In this case biocorrosion is understood to be the gradual breakdown of the material caused by substances in the body. Even with biocorrodable materials an influence on the corrosion process is advantageous.

However, in the case of biocorrodable implants complete inhibition of the corrosion should not generally be achieved, since it is ultimately desirable for the implant to dissolve in the body after a certain time. Instead, it is merely necessary to influence the rate of corrosion, which enables a delayed degradation of the implant in the body as required.

One approach in order to improve the protection against corrosion is, as also in the case of permanent implants, the application of a corrosion-inhibiting layer.

By way of example reference is made to DE 103 57 281 A1, which discloses a degradable stent made of a magnesium material, which is provided with a coating which delays the degradation. In this connection the uncoated surface of the implant, which has a natural mixed oxide layer, is transformed into a mixed fluoride layer. The coating can be produced by dipping into fluoride-containing media with or without electrolytic assistance.

The object of the present invention is to provide an alternative method for the surface treatment of a biocorrodable implant, by which the rate of degradation of the implant can be adapted as required.

This object is achieved by the method according to the invention for the surface treatment of a biocorrodable implant according to claim 1.

The method according to the invention for the surface treatment of a biocorrodable implant by means of electrochemical reactions comprises the steps of:

a) providing an implant made of a bicorrodable magnesium alloy;

b) introducing the implant into an electrolyte with a pH value of pH 9 to pH 13;

c) electrochemically treating the surface of the implant,

wherein the implant serves as the working electrode and there is also a counterelectrode, and

wherein the working electrode is alternately polarized cathodically and anodically, the current density being set to −0.1 to −75 mA/cm² for the cathodic polarization and the current density is set to 0.1 to 25 mA/cm² for the anodic polarization.

A magnesium hydride layer which grows from the implant surface into the implant is produced by the method according to the invention. Hydrogen ions are deposited cathodically from the electrolyte and are implanted into the surface of the implant.

A metal hydride layer is formed which, starting from the surface of the implant, virtually grows into the implant. Thus the method has the advantage that no change to the geometry of the implant takes place, since the metal hydride layer grows into the implant.

In the context of the present invention an implant should be understood to be an artificial material implanted in the body. Due to the use of a biocorrodable alloy for the implant body, the material used is gradually broken down by the substances in the body. It is provided that the implant consists, completely or in parts, of a biocorrodable alloy. The implants can fulfill different purposes and functions as required, such as for example interference screws, screws and plates for fixing bones, implants as medication depots, joint prostheses, stents, jaw and tooth implants. The list is merely by way of example and should in no way be understood as definitive.

The corrosion of the implant is slowed down by the hydride layer. The rate of corrosion of the hydride layer is less than that of the actual material without a hydride layer. So long as a closed hydride layer is present on the surface of the implant, the rate of corrosion of the implant is determined by the corrosion reaction of the magnesium hydride. As soon as this hydride layer is broken down by corrosion, the rate of corrosion of the implant corresponds to the rate of corrosion of the actual biocorrodable magnesium alloy. Accordingly, after the hydride layer is broken down by corrosion the alloy is broken down further, as would be the case with an untreated implant. The formation of the hydride layer gives rise to a two-stage corrosion behavior.

The method according to the invention is preferably carried out as follows:

An implant, for example a compression screw, made of a biocorrodable metal alloy, preferably a biocorrodable magnesium alloy, is threaded onto a platinum wire. Then the surface of the screw is activated by a bath in aqueous citric acid solution, preferably a 1-10% solution, for 1 to 10 seconds. Then the testpiece is rinsed in deionized water, preferably for approximately 5 to 30 seconds.

For the further treatment the screw is fixed on a non-metallic object carrier. The platinum wire is then pulled out. Indentations in the object carrier prevent later slipping of the screw. Alternatively, the screw can be inserted through a plate with a hole, wherein the ends of the screw are free in order to produce the contact later, for example with terminals.

The screw is contacted with terminals in order to produce conductive contact. For this purpose, the terminals are preferably attached to the external ends of the screw.

If very small implants such as very small screws or pins are used, no terminals are used, since contact cannot be produced by terminals. Instead a fine metal mesh is used, onto which the screws or pins are laid. The conductive contact to the implant is then produced with the aid of the mesh. The activation in the citric acid solution as well as the rinsing with water are likewise preferably carried out with the aid of the mesh.

Then the implant is introduced into the electrolyte. The electrolyte has a basic pH from pH 9 to pH 13, preferably from pH 9 to pH 10. Furthermore, it is preferable that the electrolyte contains 0.01 M NaOH and 0.2 M Na₂SO₄.

The formation of the magnesium hydride layer is made possible by the basic pH value. At a pH value below pH 9 the magnesium material would corrode because of its base metal nature.

First of all, cleaning of the surface of the implant takes place by the action of positive pulses. In this case the implant forms the working electrode. Furthermore, a counterelectrode is present in the arrangement. The counterelectrode preferably consists of a corrosion-resistant metal material, for example platinum, chromium-nickel steel, etc. Glass vessels are preferably used as electrolysis cells.

For cleaning of the surface a positive pulse of 15 mA/cm² to 35 mA/cm² is preferable with a pulse length (pulse duration) of 0.10 seconds to 0.50 s and a total duration of the pulses of overall 5 min to 40 min. In this case particular preference is given to a positive pulse of 25 mA/cm² at a pulse length of 0.20 s and a total duration of 20 min.

Then the hydrogenation of the implant takes place by negative and positive pulse changes alternating multiple times. For the method according to the invention it is preferable that the working electrode is alternately polarized cathodically and anodically multiple times, starting with a cathodic polarization and ending the deposition with a cathodic polarization.

In a preferred embodiment the current density is set to −35 to −55 mA/cm² for the cathodic polarization and the current density is set to 5 to 25 mA/cm² for the anodic polarization.

Furthermore, it is preferable that the current density and the total duration of the pulses are lower in an anodic polarization step than in a preceding anodic polarization step.

In the context of the present invention a polarization step should be understood as a succession of positive or negative pulses of a specific current density and pulse length.

Furthermore, it is preferable that the pulse length in the cathodic polarization is 0.40 s to 2.5 s and in the anodic polarization is 0.10 s to 0.50 s.

In a preferred embodiment of the method according to the invention the total duration of the pulses in a cathodic polarization step is 5 min to 90 min and the total duration of the pulses in an anodic polarization step is 1 min to 20 min.

In a further preferred embodiment the total duration of all pulses of the cathodic and anodic polarization steps is 20 min to 300 min, preferably 120 min to 240 min, particularly preferably 195 min.

In a preferred embodiment the method according to the invention consists of an alternating succession of five polarization steps:

1. Polarization step: cathodic polarization (negative pulse)

Current density: −0.1 to −75 mA/cm²

Pulse length: 0.50 to 2.5 s

Overall for 60 min (3.6 ks)

2. Polarization step: anodic polarization (positive pulse)

Current density: +0.1 to +25 mA/cm²

Pulse length: 0.20 to 0.5 s

Overall for 10 min (0.6 ks)

3. Polarization step: cathodic polarization (negative pulse)

Current density: −0.1 to −75 mA/cm²

Pulse length: 0.50 to 2.5 s

Overall for 60 min (3.6 ks)

4. Polarization step: anodic polarization (positive pulse)

Current density: +0.1 to +15 mA/cm²

Pulse length: 0.20 to 0.5 s

Overall for 5 min (0.3 ks)

5. Polarization step: cathodic polarization (negative pulse)

Current density: −0.1 to −75 mA/cm²

Pulse length: 0.50 to 2.5 s

Overall for 60 min (3.6 ks)

A deposition rate of 5 to 8 nm/h is advantageously achieved by the method according to the invention.

In the context of the present invention the deposition rate should be understood to be the growth of the metal hydride layer from the surface of the implant into the implant.

After the alternating cathodic and anodic polarization, the implant is removed from the electrolyte and rinsed for approximately 30 to 60 seconds with deionized water. For passivation the implant is introduced into a hot air stream, preferably at a temperature of 60° C. for 10 to 100 seconds. Until further use, the implant is preferably packaged in an airtight manner in order to prevent oxidation.

With the aid of the method according to the invention a magnesium hydride layer which increases the corrosion resistance of the implant is formed on the surface of the implant. Higher current intensities than those specified for the method according to the invention can actually lead to a faster formation of the hydride layer within a defined time interval. However, the faster growth of the hydride layer, which is accompanied by a different depth of penetration of the hydride layer, can lead to an inhomogeneous surface and thus to inconsistent corrosion. In the event of an inhomogeneous layer thickness of the formed magnesium hydride layer, thinner layer zones are completely broken down faster than thicker zones. If the magnesium hydride layer is already broken down on some zones, but not yet on others, this can lead to a dramatic increase in the rate of corrosion, since at these points there is no longer any hydride corrosion, but corrosion of the actual material takes place. Thus the implant is broken down inconsistently and can lose its stability.

The described parameters lead to an optimal surface in a reasonable time.

The growth of the hydride layer takes place during the cathodic polarization step. Longer or shorter pulse lengths only have an indirect effect on the growth of the hydride layer. In addition to the electrochemical reaction the pulse primarily serves the purpose that the formed hydrogen (H₂) is released uniformly and in short intervals on the working electrode. Accumulations of hydrogen bubbles can lead to slowing down or interruption of the buildup of the hydride layer at this point, since in the extreme case contact no longer takes place between the material (working electrode) and the electrolyte.

Within a polarization step there is a short resting phase (“break”) between the individual pulses. In this case this resting phase between ‘current is applied’ and ‘current is not applied’ should preferably be long enough so that the hydrogen bubbles can rise up from the working electrode.

In particular square wave pulse currents are advantageous for the method according to the invention, since they provide enough time so that the hydrogen can rise up. In the context of the present invention a square wave pulse current is a current with a steep rise and fall and a constant plateau located between them. The same applies to the pulse length. With many short pulses in a time interval, the resting phase is too short and a substantial accumulation of hydrogen gas in the form of bubbles occurs on the working electrode.

In an advantageous configuration of the method according to the invention a rest break of at least 0.1 s is provided between two pulses.

In this case the geometry of the implant (working electrode) also has an effect on the optimal pulse length. A smooth or uniform surface encourages the hydrogen bubble to drop off. The pulse length can be truncated here. Samples with an uneven surface or thread, as in the case of helical implants, or a supporting mesh as electrode, as used in the case of small implants, leads to the hydrogen bubbles requiring more time to drop off.

Thus the pulse length is adjustable according to the geometry of the implant. If too many hydrogen bubbles accumulate on the working electrode, the pulse length is made longer.

In this way the individual method parameters are adaptable to different implant sizes and geometries. Moreover, the rate of degradation of the implant can be adapted as required. If a fast degradation is desirable, the total duration of the pulses, that is to say the duration of the respective cathodic polarization step, is reduced in order to limit the formation of the hydride layer to a small depth of penetration and thus to a small layer thickness. On the other hand, in the event of a longer total duration of the pulses the depth of penetration and thus the layer thickness is increased.

In a further embodiment of the method according to the invention it is preferable that the implant provided is made of a biocorrodable magnesium alloy which has a magnesium component of at least 50%, The following composition is particularly preferred: a rare earth metal component of 2.5 to 5% by weight,

an yttrium component of 1.5 to 5% by weight,

a zirconium component of 0.1 to 2.5% by weight,

a zinc component of 0.01 to 0.8% by weight,

as well as unavoidable impurities, wherein the total content of possible contaminants is below 1% by weight and the aluminum component is less than 0.5% by weight, preferably less than 0.1% by weight

and the rest up to 100% by weight is magnesium.

Furthermore, it is preferable that the implant consists completely or in parts of a biocorrodable magnesium alloy.

Further advantages are provided by an implant with a corrosion-inhibiting coating, which is or may be obtained by the method according to the invention.

After the method according to the invention has been carried out, the surface of the implant has a hydrogenated outer layer which increases the corrosion resistance. In this case it is preferable that the corrosion-inhibiting hydride layer has a layer thickness of at least 10 nm, preferably at least 15 nm, particularly preferably 20 nm.

In a further preferred embodiment the implant provided, which is made of a biocorrodable magnesium alloy, has a magnesium component of at least 50%. The biocorrodable magnesium alloy from which the implant is manufactured has the following composition: a rare earth metal component of 2.5 to 5% by weight,

an yttrium component of 1.5 to 5% by weight,

a zirconium component of 0.1 to 2.5% by weight,

a zinc component of 0.01 to 0.8% by weight,

as well as unavoidable impurities, wherein the total content of possible contaminants is below 1% by weight and the aluminum component is less than 0.5% by weight, preferably less than 0.1% by weight

and the rest up to 100% by weight is magnesium.

Due to the low, almost negligible content of aluminum the biocorrodable magnesium alloy is suitable for the use of implants in human medicine, since aluminum is alleged to have properties which are harmful to health, such as the promotion of Alzheimer's disease or cancer.

Furthermore, it is preferable that the implant consists completely or in parts of a biocorrodable magnesium alloy.

The method according to the invention is explained in greater detail with reference to an embodiment.

### Embodiment

A round material made of the magnesium alloy ZfW 102 PM F is treated by the method according to the invention.

In this case the magnesium alloy ZfW 102 PM F consists of a rare earth metal component (including neodymium) of 4.05% by weight, the neodymium component corresponding to 2.35% by weight, an yttrium component of 1.56% by weight, a zirconium component of 0.78% by weight, a zinc component of 0.4% by weight, an aluminum component of 0.0032% by weight. The rest up to 100% by weight is magnesium.

The round material is a full cylinder with a diameter of 6 mm and a length of 3 cm. This full cylinder functions as a working electrode. A platinum electrode with a titanium core having a diameter of 6 mm and a length of 7 cm is used as the counterelectrode.

A 500 ml beaker is used as electrolysis cell. The electrolyte consists of 0.01 M NaOH and 0.2 M Na₂SO₄ and has a pH value of 9.4. The method is carried out at 24° C.

For cleaning of the surface a positive pulse of 25 mA/cm² at a pulse length of 0.20 s and a total duration of 20 min is used.

Then the hydrogenation of the round piece takes place by alternating negative and positive pulse changes. The method according to the invention is carried out in an alternating succession of five polarization steps:

1. Polarization step: cathodic polarization (negative pulse)

Current density: −50 mA/cm²3

Pulse length: 0.50 s

Overall for 60 min (3.6 ks)

2. Polarization step: anodic polarization (positive pulse)

Current density: +20 mA/cm²

Pulse length: 0.20 s

Overall for 10 min (0.6 ks)

3. Polarization step: cathodic polarization (negative pulse)

Current density: −50 mA/cm²

Pulse length: 0.50 s

Overall for 60 min (3.6 ks)

4. Polarization step: anodic polarization (positive pulse)

Current density: +10 mA/cm²

Pulse length: 0.20 s

Overall for 5 min (0.3 ks)

5. Polarization step: cathodic polarization (negative pulse)

Current density: −50 mA/cm²

Pulse length: 0.50 s

Overall for 60 min (3.6 ks)

After a total of 195 min a layer thickness of 18 nm is achieved.

The success of the treatment is determined by means of X-ray diffractometry (RDA), secondary ion mass spectrometry (SIMS) as well as determination of the free corrosion potential. An identical round material made of the magnesium alloy ZfW 102 PM F which has not been treated by the method according to the invention serves as a comparison.

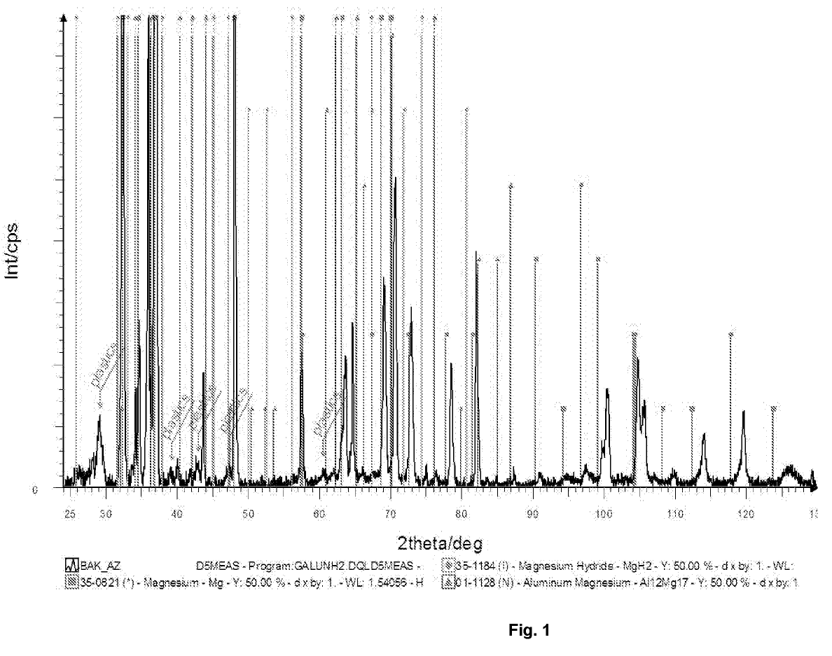

The results are presented in FIG. 1 to FIG. 4.

FIG. 1 shows the hydride detection by means of X-ray diffractometry (XRD).

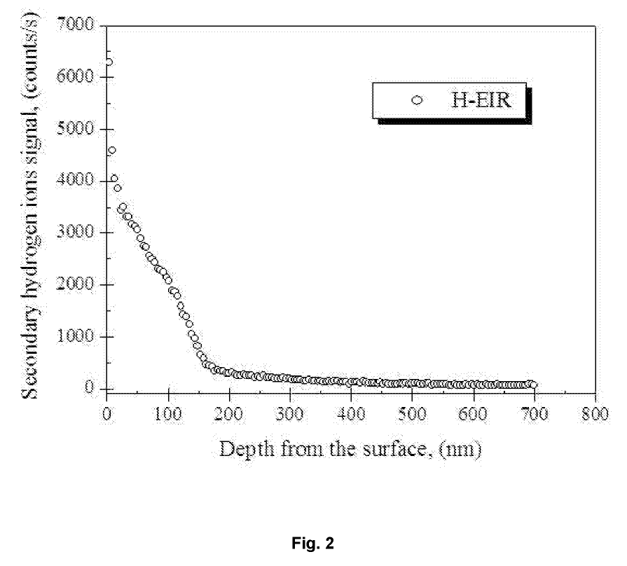

FIG. 2 shows the hydride detection by means of secondary ion mass spectrometry (SIMS),

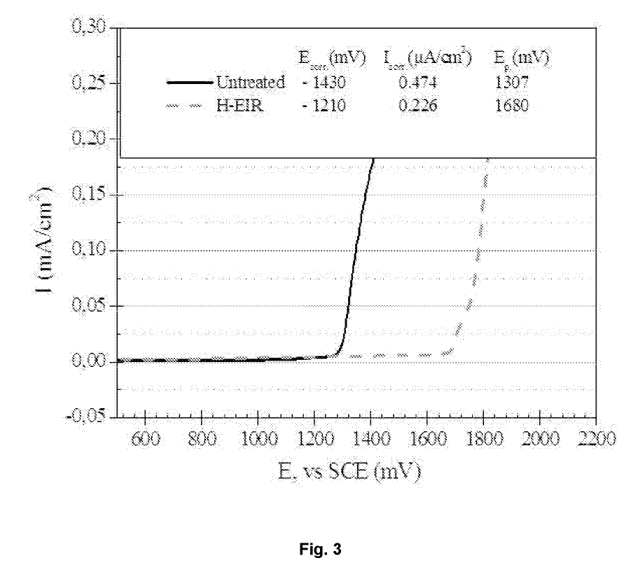

FIG. 3 shows the determination of the free corrosion potential.

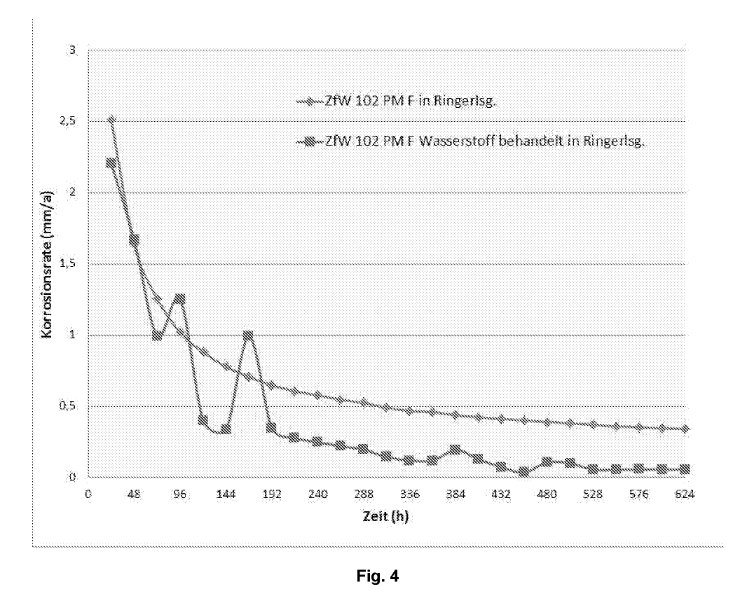

FIG. 4 shows the corrosion rate in a Ringer's lactate solution.

A round piece which had been treated by the method according to the invention according to exemplary embodiment 1 was examined by means of X-ray diffractometry. The phases present in the material are illustrated in FIG. 1. The occurrence of magnesium hydride phases (MgH₂) is evidence of the hydride layer formed by the method according to the invention.

Moreover, a round piece which had been treated by the method according to the invention according to exemplary embodiment 1 was examined by means of SIMS. FIG. 2 shows the hydride detection as a function of the depth of penetration of the hydrogen ions into the workpiece.

Moreover, the free corrosion potential of a round piece which had been treated by the method according to the invention according to exemplary embodiment 1 as well as an untreated round piece is determined. FIG. 3 shows that at 1680 mV the treated round piece (H-EIR, H electrochemical induced reaction) has a more positive corrosion potential than the untreated round piece.

FIG. 4 shows the corrosion rate of an untreated round piece and a round piece which had been treated by the method according to the invention according to exemplary embodiment 1. The corrosion rate was determined under conditions similar to those in the human body in each case at 37° C. in a Ringer's lactate solution (125-134 mmol/l Na⁺, 4.0-5.4 mmol/l K⁺, 0.9-2.0 mmol/l Ca², 106-117 mmol/l Cl^{-,} 25-31 [mmol/l] lactate). A Ringer's solution has a composition comparable to that of the blood plasma and the extracellular liquid. It can be seen that the treated round piece has a lower corrosion rate than the untreated round piece. Thus for example the untreated round piece has a corrosion rate of 0.415 mm/year after 432 h and a corrosion rate of 0.339 mm/year after 624 h, and on the other hand the round piece treated by the method according to the invention according to exemplary embodiment 1 has a corrosion rate of 0.224 mm/year after 432 h and a corrosion rate of 0.153 mm/year after 624 h (cf. FIG. 4).

Thus because of the slower rate of degradation, after implantation into the human body a biocorrodable implant which is treated by the method according to the invention has a longer service life than an untreated implant of the same structural design. Thus with the aid of the method according to the invention the rate of degradation can be adapted to the particular purpose and to the necessary residence time of the implant in the body. If it is necessary to have a longer residence time in the body than the actual material permits, the corrosion resistance can be increased by the treatment of an implant by the method according to the invention. Moreover, the increased corrosion resistance gives the implant an increased stability, since corrosion is accompanied by a loss of mass of the implant. If the implant breaks down too quickly in the body, in some circumstances the bone does not have sufficient time to grow into the implant and to replace the material by bone material. Thus the choice of the corrosion resistance is dependent upon the position of the implant in the body or also dependent upon the patient. Thus in the case of older people, who exhibit slower bone growth, a biocorrodable implant with a substantially slower rate of degradation can be used. On the other hand, if only a small implant into the bone is used, which is not subject to substantial mechanical stresses, an implant with a smaller hydride layer thickness can be used.

1. A method for the surface treatment of a biocorrodable implant by means of electrochemical reactions comprising the steps of:
a) providing an implant made of a bicorrodable magnesium alloy;
b) introducing the implant into an electrolyte with a pH value of pH 9 to pH 13;
c) hydrogenation of the implant electrochemically treating the surface of the implant,
wherein the implant serves as the working electrode and there is also a counterelectrode, and
wherein the working electrode is alternately polarized cathodically and anodically with a pulsed voltage, the current density being set to −0.1 to −75 mA/cm² for the cathodic polarization and the current density is set to 0.1 to 25 mA/cm² for the anodic polarization, and
wherein the total duration of the pulses in a cathodic polarization step is 5 min to 90 min and the total duration of the pulses in an anodic polarization step is 1 min to 20 min.

2. The method according to claim 1, characterized in that the working electrode is alternately polarized cathodically and anodically multiple times, starting with a cathodic polarization and ending the deposition with a cathodic polarization.

3. The method according to claim 1 or 2, characterized in that the current density and the total duration of the pulses are lower in an anodic polarization step than in a preceding anodic polarization step.

4. The method according to one of the preceding claims, characterized in that the pulse length in the cathodic polarization is 0.40 s to 2.5 s and in the anodic polarization is 0.10 s to 0.50 s.

5. The method according to one of the preceding claims, characterized in that the total duration of all pulses is 20 min to 300 min.

6. The method according to one of the preceding claims, characterized in that a hydride layer having a hydride layer thickness of at least 10 nm, preferably at least 15 nm is achieved on the surface of the implant.

7. An implant obtained by the method according to one of claims 1 to 6 consisting of a biocorrodable magnesium alloy and having a corrosion-inhibiting coating,
wherein the corrosion-inhibiting coating consists of a hydride layer having layer thickness of at least 10 nm, preferably at least 15 nm, and
the biocorrodable magnesium alloy contains
a rare earth metal component without yttrium of 2.5 to 5% by weight,
an yttrium component of 1.5 to 5% by weight,
a zirconium component of 0.1 to 2.5% by weight,
a zinc component of 0.01 to 0.8% by weight,
as well as unavoidable impurities, wherein the total content of possible contaminants is below 1% by weight and the aluminum component is less than 0.5% by weight, and the rest up to 100% by weight is magnesium.
